(19) United States
(12) Patent Application Publication
(10) Pub. No.: US 2022/0273573 A1
(43) Pub. Date: Sep. 1, 2022

(54) SOLID DISPERSION GENISTEIN COMPOSITIONS AND METHODS OF MAKING AND USING THE SAME

(71) Applicant: Humanetics Corporation, Minneapolis, MN (US)

(72) Inventors: Michael D. Kaytor, Maplewood, MN (US); Adam J. Harvey, Blaine, MN (US); John C. Dykstra, Edina, MN (US); Samuel Elliott Kennedy, Bend, OR (US); April Naab, Bend, OR (US); Michael Edward Grass, Bend, OR (US)

(21) Appl. No.: 17/664,015

(22) Filed: May 18, 2022

Related U.S. Application Data
(63) Continuation of application No. PCT/US2021/055271, filed on Oct. 15, 2021.
(60) Provisional application No. 63/092,838, filed on Oct. 16, 2020.

(51) Int. Cl. A61K 9/16; A61K 31/352

(52) U.S. Cl. CPC A61K 9/1635; A61K 31/352

(57) ABSTRACT

The present invention is directed to solid dispersion formulations comprising genistein and one or more pharmaceutically acceptable excipients. In particular embodiments, the one or more pharmaceutically acceptable excipients include polyvinylpyrrolidone. Various techniques can be used to form the solid dispersions, including spray drying and extrusion techniques.

### RELATED APPLICATIONS

This application is a continuation of PCT/US2021/055271, filed Oct. 15, 2021, and titled SOLID DISPERSION GENISTEIN COMPOSITIONS AND METHODS OF MAKING AND USING THE SAME, which claims priority to U.S. Provisional Patent Application No. 63/092,838, filed Oct. 16, 2020, and titled SOLID DISPERSION GENISTEIN COMPOSITIONS AND METHODS OF MAKING AND USING THE SAME, each of which is incorporated herein by reference in its entirety.

### STATEMENT REGARDING FEDERALLY SPONSORED RESEARCH OR DEVELOPMENT

This invention was made with the support under the following government contracts: W81XWH-17-1-0584 and W81XWH-19-2-0060, awarded by the Department of Defense. The government has certain rights in the invention.

### TECHNICAL FIELD

The present disclosure relates to compositions including genistein and methods for producing and utilizing such compositions. More particularly, the present disclosure relates to solid dispersion compositions including genistein and one or more pharmaceutically acceptable excipients.

### BACKGROUND

Genistein is a pharmaceutically active isoflavone. In the body, genistein interacts with various proteins that have wide-ranging actions in many tissues. Therefore, the potential therapeutic impacts of genistein are diverse. However, genistein has proven difficult to formulate and deliver to subjects in a manner that achieves and maintains a therapeutic effect.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 shows the pharmacokinetics of a solid dispersion genistein formulation versus the pharmacokinetics of comparative genistein formulations in mice.

FIG. 2 shows the thirty-day survival rates in groups of mice receiving a solid dispersion genistein formulation versus the thirty-day survival rates in groups of mice receiving comparative genistein formulations.

FIG. 3 shows the 180-day survival rates in groups of mice receiving a solid dispersion genistein formulation versus the 180-day survival rates in groups of mice receiving comparative genistein formulations.

FIG. 4 shows the pharmacokinetics of an extruded solid dispersion genistein formulation vs the pharmacokinetics of a comparative genistein formulation in nonhuman primates.

FIGS. 5A-5C show the pharmacokinetics of various dose levels of an extruded solid dispersion genistein formulation containing sucralose versus the pharmacokinetics of equivalent dose levels of synthetic genistein oral capsules in humans.

### DETAILED DESCRIPTION

Compositions of genistein are described herein. In certain embodiments, the compositions include genistein in a solid dispersion formulation. The solid dispersion formulations can also include one or more pharmaceutically acceptable excipients, such as polyvinylpyrrolidone or other water soluble polymers. Use of a water soluble polymer such as polyvinylpyrrolidone as a pharmaceutically acceptable excipient in the solid dispersion formulations can serve to increase the bioavailability of the genistein and may additionally facilitate administration or consumption of the genistein at amounts sufficient to achieve a desired nutritional or therapeutic benefit.

The solid dispersion formulations can be provided as particulates (e.g., a powder) or another solid dosage form. If desired, the particulates can be suspended in a pharmaceutically acceptable liquid prior to oral administration or consumption. The solid dispersion formulations can also be provided in other solid dosage forms, such as capsules, tablets, and the like. In certain embodiments, the solid dispersion formulations are suitable for oral administration to or consumption by a subject as a pharmaceutical formulation, a medical food, or a dietary supplement.

Methods for preparing the solid dispersion formulations are also provided herein. In some embodiments, the solid dispersion formulations are formed using spray drying techniques. In other embodiments, the solid dispersion formulations are formed using extrusion techniques. Milling techniques can also be used to obtain desired particle sizes. Other methods for preparing solid dispersion formulations are also contemplated.

Methods of treating subjects at risk for or suffering from various diseases and disorders suitable for treatment using genistein are also described herein.

### I. Definitions

It should be noted that as used herein and in the appended claims, the singular forms “a,” “an,” and “the” include plural reference unless the context clearly dictates otherwise.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed. All ranges also include both endpoints.

As used herein, “solid dispersion” refers to a group of two or more different components in a solid state. As set forth below, the solid dispersion formulations disclosed herein can include a dispersion of one or more active pharmaceutical agents in a matrix of one or more excipients. Other components can also optionally be included. The term “amorphous” refers to a lack of long-range order of molecules in the material, which can be indicated by a lack of sharp peaks in a diffractogram. An amorphous solid dispersion can also refer to two or more molecules in a stable disordered state, lacking a crystal structure.

The term “radioprotective agent” refers to agents that protect cells or living organisms from the deleterious cellular effects that result from exposure to ionizing radiation (gamma, neutron, proton, x-ray, etc.). These deleterious cellular effects include damage to cellular DNA, such as DNA strand break, disruption in cellular function, inflammation, cell death and/or carcinogenesis. More particularly, the hematopoietic system is a rapidly dividing system and is therefore centrally affected by exposure to high-dose whole body ionizing radiation. Bone marrow aplasia and the resultant leukopenia, erythropenia and thrombocytopenia predispose the animal or human to infection, hemorrhage and ultimately death. For purposes of the present disclosure, a radioprotective agent may be one that is administered prophylactically prior to potential radiation exposure, with such administration resulting in the prevention, reduction in severity, or slowing of the symptoms or effects of exposure to ionizing radiation, should such an exposure occur. Additionally, the radioprotective agent may be administered for use as a mitigator (after exposure to ionizing radiation but prior to symptoms) with such administration resulting in mitigation (i.e., prevention, reduction in severity, slowing, halting, or reversal of symptoms or effects that are otherwise associated with exposure to a given dose of ionizing radiation). Further, the radioprotective agent may be administered for use as a therapeutic (after exposure to ionizing radiation and after the presence of one or more symptoms).

A “subject” for purposes of this disclosure is an animal to which a formulation as described herein can be administered in order to achieve a therapeutic effect. In one embodiment, the subject is a human being.

“Therapeutically effective” refers to an amount of genistein or an amount of a solid dispersion formulation of genistein as described herein which achieves a therapeutic effect by inhibiting a disease or disorder in a patient or by prophylactically inhibiting or preventing the onset of a disease or disorder. A therapeutically effective amount may be an amount which relieves to some extent one or more symptoms of a disease or disorder in a patient; returns to normal either partially or completely one or more physiological or biochemical parameters associated with or causative of the disease or disorder; and/or reduces the likelihood of the onset of the disease or disorder.

### II. Genistein Compositions

Genistein is one of several known isoflavones that are normally found in plants. The main sources of natural genistein are soybeans and other legumes. Genistein is commercially available and may be obtained in synthetic, purified form. Synthetic genistein is available, for example, as BONISTEIN. Genistein's chemical name is 5,7-dihydroxy-3-(4-hydroxyphenyl)-chromen-4-one (IUPAC). Genistein is an active pharmaceutical agent and has the following chemical structure:

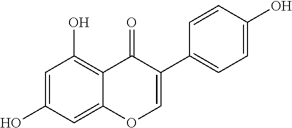 [SMILES: O=c1c(-c2ccc(O)cc2)coc2cc(O)cc(O)c12]

The genistein compositions described herein include genistein in solid dispersion formulations. The solid dispersion formulations include genistein and one or more pharmaceutically acceptable excipients. If desired, the solid dispersion formulations can optionally include one or more additional components or additives including, but not limited to, fillers, preservatives, colorants, flavorants, sweeteners, dispersants, antistatic agents, glidants, other processing aides (e.g., plasticizers), and the like. The additives can be added during manufacture of the solid dispersion formulations, or during a post processing step after the solid dispersion formulations have been formed.

In some embodiments, the genistein is dispersed substantially uniformly throughout the matrix of the pharmaceutically acceptable excipient that forms the solid dispersion. The amount of genistein in the solid dispersion can vary. For example, in particular embodiments, the solid dispersion formulations include genistein at a concentration of between about 25% and about 50% (w/w), between about 25% and about 45% (w/w), or between about 30% and about 40% (w/w).

Various types of pharmaceutically acceptable excipients can be used. In some embodiments, the pharmaceutically acceptable excipients include one or more water soluble polymers. Water soluble polymers include pharmaceutically acceptable polymers that can be dissolved or dispersed in water. Suitable water soluble polymers for use in the solid dispersion formulations described herein may be selected from, for example, vegetable gums, such as alginates, pectin, guar gum, and xanthan gum, modified starches, polyvinylpyrrolidone (PVP), polyvinylpyrrolidone-co-vinylacetate (PVPVA), hypromellose (HPMC), methylcellulose, and other cellulose derivatives, such as sodium carboxymethylcellulose, hydroxypropylcellulose, and the like. In particular embodiments, the solid dispersion formulations include polyvinylpyrrolidone as a water soluble polymer.

In some embodiments, the total content of the one or more pharmaceutically acceptable excipients in the solid dispersion ranges from between about 50% and about 75% (w/w), between about 55% and about 75%, or between about 60% and about 70% (w/w). For instance, in a particular embodiment, the solid dispersion comprises between about 50% and about 75%, (w/w) or between about 60% and about 70% (w/w) of a water soluble polymer such as polyvinylpyrrolidone.

As previously discussed, the solid dispersion formulations can optionally include one or more additional components or additives such as fillers, preservatives, colorants, flavorants, sweeteners, dispersants, antistatic agents, glidants, other processing aides (e.g., plasticizers), and the like. In one embodiment, the solid dispersion formulations optionally include a non-nutritive sweetener such as sucralose, aspartame, saccharin, stevia, and the like. Other non-nutritive or nutritive sweeteners (e.g., dextrose, fructose, sucrose, and the like) can also be used. As previously discussed, the additives can be added during the manufacture of the solid dispersion formulations, or during a post processing step after the solid dispersion formulations have been formed. For instance, in some embodiments a sweetener is added after the solid dispersion formulations have been formed.

The solid dispersion formulations can be formed in various ways. In certain embodiments, the solid dispersion formulations are formed using spray drying techniques. When employing spray drying techniques, a mixture of genistein, the one or more pharmaceutically acceptable excipients (e.g., polyvinylpyrrolidone), and the one or more optional additives can be solubilized in a solvent to form a solution or suspension. Exemplary solvents include, but are not limited to, organic solvents such as dimethyl sulfoxide (DMSO), dimethyl formamide (DMF), methanol, ethanol, acetone, dichloromethane, and combinations thereof. The solution or suspension is then spray dried, resulting in an amorphous solid dispersion of genistein dispersed in a matrix of the one or more pharmaceutically acceptable excipients (e.g., polyvinylpyrrolidone) and the one or more optional additives. The one or more optional additives can also be blended after the amorphous solid dispersion has been spray dried and formed. In certain embodiments, the amorphous solid dispersion includes a substantially uniform distribution of genistein throughout the matrix.

The particle or particulate size of the spray dried amorphous solid dispersion can vary depending on the parameters of the spray drying technique. In some embodiments, the particle or particulate size of the spray dried amorphous solid dispersion is between about 1 micron and about 1000 microns, or between about 10 microns and about 1000 microns. Larger or smaller particles or particulates can also be formed, such as particles or particulates between about 1 micron and about 10 microns, between about 1 micron and about 100 microns, between about 100 microns and about 300 microns, between about 300 microns and about 600 microns, or between about 600 microns and about 1000 microns. Other sizes are also contemplated.

In other embodiments, the solid dispersion formulations are formed by extrusion techniques, such as hot melt extrusion techniques. When employing hot melt extrusion techniques, a mixture of genistein particles, the one or more pharmaceutically acceptable excipients (e.g., polyvinylpyrrolidone), and the one or more optional additives can be heated to a melt temperature of between about 140° C. and about 220° C., or between about 160° C. and about 200° C. As the mixture is heated, the genistein particles and/or the one or more pharmaceutically acceptable excipients are melted or otherwise softened to produce a blend of genistein dispersed in the one or more melted or softened pharmaceutically acceptable excipients and one or more optional additives. The blend can then be extruded to form an amorphous solid dispersion of genistein dispersed in a matrix of the one or more pharmaceutically acceptable excipients (e.g., polyvinylpyrrolidone) and one or more optional additives. The one or more optional additives can also be blended after the amorphous solid dispersion has been extruded and formed. For instance, one or more optional additives can be added to the composition after milling the extruded amorphous solid dispersion. In certain embodiments, the solid dispersion includes a substantially uniform distribution of genistein throughout the matrix. Further, in some embodiments, the melt temperature of the extrusion is at least 50° C., 60° C., 70° C., 80° C., 90° C., 100° C., or 110° C. lower than the melting point of the genistein. In yet further embodiments, the melt temperature of the extrusion is between about 50° C. and about 150° C., or between about 100° C. and about 130° C. lower than the melting point of the genistein.

The extruded solid dispersion can be in various forms. In some embodiments, the extruded solid dispersion is in pellet or rod form. If desired, the extruded solid dispersion can be milled into smaller amorphous solid dispersion particles or particulates. In some embodiments, the extruded solid dispersion is milled into particles or particulates of between about 1 micron and about 1000 microns, or between about 10 microns and about 1000 microns. Larger or smaller particles or particulates can also be formed, such as particles or particulates between about 1 micron and about 10 microns, between about 1 micron and about 100 microns, between about 100 microns and about 300 microns, between about 300 microns and about 600 microns, or between about 600 microns and about 1000 microns. Other sizes are also contemplated.

In certain embodiments, the particles or particulates of the solid dispersion formulations disclosed herein can be prepared for oral consumption via a variety of delivery devices or mechanisms. For example, the particles or particulates of the solid dispersion formulations can be prepared for delivery from any desired metering device, including a measuring spoon, cup, or vial. In yet further embodiments, the particles or particulates of the solid dispersion formulations can be metered in pre-measured amounts into packets. In some of such embodiments, the particles or particulates of the solid dispersion formulations can also be mixed with a pharmaceutically acceptable liquid prior to or during use. For instance, the particles or particulates of the solid dispersion formulations can be mixed with water or another pharmaceutically acceptable liquid prior to being orally administered or otherwise consumed. For example, the particles or particulates of the solid dispersion formulations can be metered in pre-measured amounts into packets. Prior to consumption, a subject may mix the particles or particulates from one or more packets with a pharmaceutically acceptable liquid. In some embodiments, one or more suspending agents are also included with the formulation to aid in suspending the particles or particulates in the pharmaceutically acceptable liquid. This mixture can thereafter be consumed. The particles or particulates of the solid dispersion formulations can also be mixed with another food or pharmaceutical agent, or they can be consumed separately. The particles or particulates of the solid dispersion formulations can also be referred to as a powder.

In further embodiments, the particles or particulates of the solid dispersion formulations can be formulated into capsules or tablets. For instance, capsules can include a pharmaceutically acceptable casing or shell in which the particles or particulates of the solid dispersion are disposed. In some embodiments, one or more pharmaceutically acceptable excipients can optionally be included in the capsules or tablets. In other embodiments, the capsules or tablets do not contain additional pharmaceutically acceptable excipients. Edible and/or chewable tablets comprising the solid dispersion formulations are also contemplated. In some embodiments, one or more binding agents can be used to aid in forming the edible and/or chewable tablets.

The inventors have also found that solid dispersion formulations prepared according to the present description can increase bioavailability of genistein relative to other types of genistein formulations. In particular, as is illustrated in the experimental examples that follow, solid dispersion formulations prepared as described herein exhibited significantly improved relative bioavailability when compared to aqueous suspension formulations, oil suspension formulations, and crystallized formulations comprising genistein. Such a result is surprising and would not be generally expected. For example, in certain embodiments, the solid dispersion formulations prepared according to the present description provide an increase in peak total genistein serum concentration of greater than about 200%, 300%, 400%, 500%, 600%, or 700% compared to aqueous suspension formulations, oil suspension formulations, and crystallized formulations comprising genistein.

The significantly increased relative bioavailability provided by the solid dispersion formulations described herein presents several advantages. For example, the increase in bioavailability afforded by the solid dispersion formulations described herein provides the added benefit of reducing the amount of genistein that must be delivered to a subject in order to achieve and maintain therapeutic genistein blood plasma levels. Therefore, the formulations described herein can offer a significant reduction in the relative amount of administered genistein required to achieve and maintain a therapeutic benefit, which can reduce the costs of genistein treatments, work to mitigate or avoid potential side effects that may be associated with relatively higher doses of the compound, and further decreases the amount of formulated drug substance required to achieve and maintain therapeutic efficacy. The formulations described herein are also suitable for oral consumption rather than injection, providing a significant advantage for methods of administration.

### III. Methods

The solid dispersion formulations described herein can be used to treat subjects suffering from or at risk for a disease or disorder treatable with genistein. Clinical trials, animal studies, cell-culture experiments, and epidemiological studies have provided evidence that genistein exerts various physiological effects. Examples of diseases and disorders amenable to treatment by genistein are described herein. However, the potential therapeutic applications of genistein are not limited to those described herein, and genistein formulations according to the present description can be used to treat a subject at risk for or suffering from any disease or disorder for which administration of genistein will be therapeutically effective. For instance, the genistein formulations disclosed herein can be used in the treatment of inflammatory lung diseases associated with respiratory viruses, including, but not limited to, the viruses associated with coronavirus disease 2019 (COVID-19), severe acute respiratory syndrome (SARS), middle ease respiratory syndrome (MERS), and the like. In some embodiments, the genistein formulations disclosed herein can be prepared for intranasal administration. For instance, the genistein formulations can be prepared for administration via aerosolization and inhalation, or through the use of nebulization for use as a pulmonary formulation.

As one example, genistein has displayed antitumor, antimetastatic and antiangiogenic (suppression of blood-vessel growth) properties in tissue culture and in vivo. Several epidemiological studies suggest that soybean consumption may contribute to lower incidence of breast, colon, prostate, thyroid, and head and neck cancers—an effect that is attributed to genistein and other isoflavones (Takimoto et al., Cancer Epidemiol Biomarkers Prev. 2003 November; 12(11 Pt 1): 1213-21; Wei et al., J Nutr. 2003 November; 133(11 Suppl 1): 3811S-3819S; Magee P. J. and I. R. Roland, Br J Nutr. 2004 April; 91(4): 513-31; Park, 0. J. and Y. J. Surh, Toxicol Lett. 2004 Apr. 15; 150(1): 43-56; Messina, M. J., Nutr Re. 2003 April; 61(4): 117-31). Genistein has also been reported to inhibit non-Hodgkin's lymphoma, melanoma, lung cancers, and ovarian cancer (Wei et al. 2003; 2(12): 1361-8; Nicosia et al., Hematol Oncol Clin North Am. 2003 August; 17(4): 927-43; Sun et al., Nutr Cancer. 2001; 39(1): 85-95). Tissue culture experiments suggest that genistein's cancer-fighting effects occur at dosages that are hard to attain from food alone, unless one eats very large amounts of soy products. Reliable genistein dosing therefore requires the use of concentrated supplements (Magee and Roland 2004).

The solid dispersion formulations may, therefore, be used in methods of inhibiting the onset, development, progression, or treatment related sequelae of certain cancers, such as cancers selected from breast, colon, prostate, thyroid, and head and neck cancers. In one such embodiment, a subject at risk for developing a breast, colon, prostate, lung, thyroid, head or neck cancer is identified and a therapeutically effective amount of a solid dispersion formulation selected from any of those described herein is administered to the subject. The solid dispersion formulations described herein may also be used in methods of treating cancer. In a particular embodiment, a patient at risk for or suffering from a cancer responsive to genistein treatment, such as for example, a cancer selected from non-Hodgkin's lymphoma, melanoma, lung cancers, and ovarian cancer is identified and a therapeutically effective amount of a solid dispersion formulation selected from any of those described herein is administered to the subject.

The ability of genistein and related soy isoflavones to reduce post-menopausal bone-loss has also been shown in many studies. These substances prevent bone loss and promote bone formation, especially in the spine. Among the dosage regimens found to be effective are: 1 mg/day genistein+0.5 mg/day daidzein+42 mg/day other isoflavones (biochanin A and formononetin, in this case); 54 mg/day genistein; 57 mg/day isoflavones; 65 mg/day isoflavones; 90 mg/day isoflavones (Morabito et al. J Bone Miner Res. 2002 October; 17(10); 1904-12; Cotter A. and K. D. Cashman, Nutr Rev. 2003 October; 61(10): 346-51; Atkinson et al., Am J Clin Nutr. 2004 February; 79(2): 326-33; Setchell K. D. and E. Lydeking-Olsen, Am J Clin Nutr. 2003 September; 78(3 Suppl); 593S-609S; Clifton-Bligh et al., Menopause. 2001 July-August; 8(4): 259-65; Fitzpatrick, L. A., 2003 Mar. 14; 44 Supl 1: S21-9). Therefore, methods for reducing post-menopausal bone-loss are also provided herein. In one embodiment, such a method includes identifying a subject at risk for or suffering from post-menopausal bone loss and administering to the subject a therapeutically effective amount of a solid dispersion formulation selected from any of those described herein. Alternatively, methods for promoting bone formation are also provided. In one such embodiment, a method for promoting bone formation, such as in the spine, includes identifying a subject at risk for or suffering from loss of bone mass and administering to the subject a therapeutically effective amount of a solid dispersion formulation selected from any of those described herein.

Genistein has also been suggested for use in treating cystic fibrosis. The main clinical symptoms of cystic fibrosis are chronic obstructive lung disease, which is responsible for most of the morbidity and mortality associated with cystic fibrosis, and pancreatic insufficiency. Cystic fibrosis (CF) is caused by a mutation in the cystic fibrosis transmembrane conductance regulator (CFTR), a plasma membrane protein. CFTR functions as a chloride channel, and about 1000 mutations of the gene coding for CFTR are currently known. The most common of these known mutations results in a deletion of a phenylalanine at position 508 of the CFTR protein. This mutation is referred to as Delta508 and is present in the majority of patients suffering from cystic fibrosis. The Delta508 mutation results in an aberrant CFTR that is not transported to the plasma membrane, but is instead degraded in the ubiquitin-proteasome pathway. One approach for developing a treatment for cystic fibrosis is to inhibit the breakdown of DeltaF508-CFTR by interfering with the chaperone proteins involved in the folding of CFTR. Genistein has been shown in in vitro systems to inhibit the breakdown of DeltaF508-CFTR through interference with the relevant chaperone proteins. In addition, it has been shown that it is possible to stimulate CFTR or its mutated forms, when present in the plasma membrane, using genistein (Roomans, G. M., Am J Respir Med. 2003; 2(5): 413-31).

The solid dispersion formulations described herein may be used in treating cystic fibrosis. In an embodiment of such a method, a subject at risk for or suffering from cystic fibrosis is identified and a therapeutically effective amount of a solid dispersion formulation selected from any of those described herein is administered to the subject. In a particular embodiment, a subject at risk for or suffering from cystic fibrosis associated with DeltaF508-CFTR is identified and a therapeutically effective amount of a solid dispersion formulation selected from any of those described herein is administered to the subject. In each embodiment of a method for treating cystic fibrosis described herein, the therapeutically effective amount of solid dispersion formulation administered to the subject is sufficient to accomplish one or more of the following: inhibit the breakdown of DeltaF508-CFTR; inhibit or prevent the onset of cystic fibrosis or one or more symptoms associated with cystic fibrosis; mitigate or reduce the severity of one or more symptoms associated with cystic fibrosis; delay the progression of cystic fibrosis or the worsening of one or more symptoms associated with cystic fibrosis.

Genistein appears to increase the rate at which fats are metabolized by the body, and to decrease the rate at which they are deposited in the tissues (Goodman-Gruen, D. and D. Kritz-Silverstein, Menopause. 2003 September-October; 10(5): 427-32). Moreover, in clinical studies of humans and animals, the consumption of genistein and daidzein resulted in loss of body fat, lower fasting insulin concentrations, lower LDL and higher HDL cholesterol, and improved insulin responses to blood sugar. Cholesterol benefits were seen at dosages of 42 mg/day of genistein plus 27 mg/day of daidzein (Bhathena, S. J. and M. T. Velasquez, Am J Clin Nutr. 2002 December; 76(6): 1191-201; Urban et al., J Urol. 2001 January; 165(1): 294-300). In addition to lowering LDL and raising HDL (mentioned above), genistein prevents the oxidation of LDL, a process thought to contribute to arterial plaques (Young, S. G. and S. Parthasarathy, West J Med. 1994 February; 160(2): 153-54). The solid dispersion formulations described herein can be used in methods for lowering LDL and/or raising HDL in subjects in need thereof. In one such embodiment, a subject at risk for or suffering from a high circulating level of LDL is identified and a therapeutically effective amount of a solid dispersion formulation selected from any of those described herein is administered to the subject, wherein the therapeutically effective amount of solid dispersion formulation is sufficient to lower the LDL levels or prevent or delay an increase in circulating LDL levels in the subject. In another embodiment, a subject that could benefit from an increase in circulating levels HDL is identified and a therapeutically effective amount of a solid dispersion formulation selected from any of those described herein is administered to the subject, wherein the therapeutically effective amount of solid dispersion formulation is sufficient to increase circulating HDL levels or prevent or delay decrease in circulating HDL levels in the subject.

Genistein is also a radioprotective agent. For example, genistein has been reported to increase hematopoiesis and survival in irradiated mice (Zhou, 2005; Landauer, 2001, 2003 & 2005). The mechanism of action for this radioprotective effect may potentially involve several of genistein's known effects including inhibition of pro-inflammatory cytokines, inhibition of protein tyrosine kinases (PTKs) and PTK-triggered apoptosis, inhibition of topoisomerase II, inhibition of phosphatidylinositol turnover and the second messenger system, both agonist and antagonist estrogenic effects, reduction of stress gene expression through inactivation of Y/CCA-AT binding factor, increased antioxidant activity, apoptosis, cell cycle arrest and differentiation, improved immune defenses and/or increased AKT kinase levels. The beneficial effects of genistein may also be due, in part, to its antioxidant properties, reducing free radicals and stabilizing the cell membrane structure. Further, genistein may also have a role in protecting stem cells and/or stimulating proliferation.

Genistein administered prior to, during, and/or after exposure to radiation, may be used to eliminate or reduce the severity of deleterious cellular effects caused by exposure to ionizing radiation resulting from, for example, from a nuclear explosion, a spill of radioactive material, close proximity to radioactive material, cancer radiation therapy, diagnostic tests that utilize radiation, and the like. Genistein can be used for the treatment and prevention of Acute Radiation Syndrome (ARS) (sometimes known as radiation toxicity or radiation sickness) or the delayed effects of acute radiation exposure (DEARE). ARS is an acute illness caused by irradiation of a substantial portion of the body by a high dose of penetrating radiation (i.e., greater than 0.7 Gray (Gy) or 70 rads, with mild symptoms possible at doses as low as 0.3 Gy or 30 rads) over a very short period of time (usually a matter of minutes). It is thought that the major cause of ARS is depletion of immature parenchymal stem cells in specific tissues. DEARE is also caused by irradiation of a substantial portion of the body by a high dose of penetrating radiation. However, DEARE pathology manifests in survivors of ARS as late effects that may occur weeks to months after radiation exposure. DEARE causes chronic illness that can impact multiple organ systems. For instance, DEARE can cause illnesses such as pneumonitis and/or pulmonary fibrosis.

Methods for treating radiation exposure are, therefore provided herein. In each embodiment, a subject at risk of or that has suffered from exposure to radiation is identified and a therapeutically effective amount of a solid dispersion formulation selected from any of those described herein is administered to the subject. In specific embodiments, the method of treating radiation exposure is a method for preventing or mitigating ARS or DEARE, wherein a subject at risk of ARS or DEARE is identified and a therapeutically effective amount of a solid dispersion formulation as described herein is administered to the subject before the subject is exposed to radiation. In other embodiments, the method of treating radiation exposure is a method for treating ARS or DEARE, wherein a subject suffering from ARS or DEARE is identified and a therapeutically effective amount of a solid dispersion formulation as described herein is administered to the subject after the subject has suffered exposure to radiation. In yet other embodiments, a subject at risk of radiation exposure is identified, a therapeutically effective amount of a solid dispersion formulation as described herein is administered to the subject prior to exposure to radiation, and, in the event the subject suffers from radiation exposure, administration of therapeutically effective amounts of genistein is continued after the radiation exposure occurs.

In additional embodiments, subjects at risk for or having suffered from a radiation exposure resulting from an event selected from cancer radiation therapy or a diagnostic test utilizing radiation are identified, and the subjects are administered a therapeutically effective amount of the solid dispersion formulation. In one such embodiment, the solid dispersion formulation is administered to the subject prior to radiation exposure in order to prevent or reduce the severity of the deleterious effects of such exposure. In another such embodiment, the solid dispersion formulation is administered to the subject after radiation exposure in order to mitigate, reverse or reduce the severity of the deleterious effects of such exposure. In still another embodiment, the methods of treating radiation exposure resulting from an event selected from cancer radiation therapy or a diagnostic test utilizing radiation in a subject may include administering a solid dispersion formulation as described herein both before and after radiation exposure.

In each of the embodiments of the methods described herein, the therapeutically effective amount of solid dispersion formulation may be administered orally. Where the formulation is administered orally, the formulation may be prepared in any manner suitable for oral administration, such as is described herein. The dose and dosing regimen most appropriate for a given embodiment of the therapeutic methods described herein may depend upon, for example, the subject being treated, the nature of the disease or disorder, as well as the severity of any symptoms suffered. Using formulations prepared as described herein, one of skill in the art will be able to identify the appropriate dose and dosing regimen useful for achieving therapeutic efficacy in each of the methods described herein. The solid dispersion formulations described herein may be administered, for example, as a single dose, a regular daily dose, a two-times daily dose, a three-times daily dose, or according to another desired dosing schedule. In some embodiments, the solid dispersion formulations are administered prophylactically, such as daily for at least (or up to) 1 day, 2 days, 3 days, 4 days, 5 days, 6 days, 7 days, 8 days, 9 days, 10 days, 11 days, 12 days, 13 days, or 14 days prior to anticipated exposure to radiation. In certain embodiments, the solid dispersion formulations are administered prophylactically, such as daily between about 1 day and about 14 days, between about 1 day and about 10 days, or between about 1 day and about 7 days prior to anticipated exposure to radiation. The solid dispersion formulations can also be administered daily after exposure to radiation (or after symptoms associated with exposure to radiation) for at least (or up to) about 4 weeks, about 5 weeks, about 6 weeks, about 7 weeks, about 8 weeks, about 9 weeks, about 10 weeks, about 11 weeks, or about 12 weeks. In some instances, longer administration of the solid dispersion formulations is also contemplated, such as for at least 4 months, 5 months, 6 months, or longer. In still other embodiments, the solid dispersion formulations are administered daily after exposure to radiation (or after symptoms associated with exposure to radiation) for between about 4 weeks and about 6 months, 4 weeks and about 5 months, 4 weeks and about 3 months, 4 weeks and about 12 weeks, or between about 6 weeks and about 10 weeks. Other dosing regimens are also contemplated.

The total daily dose of genistein delivered using a formulation or method described herein may depend on the desired condition to be treated or the desired therapeutic effect. In specific embodiments, a therapeutically effective amount of a solid dispersion formulation according to the present description may be an amount sufficient to deliver a dose of genistein ranging from about 50 mg/day to about 10,000 mg/day. In certain such embodiments, the amount of solid dispersion formulation administered to the subject is sufficient to deliver a dose of genistein selected from about 50 mg/day to about 9,000 mg/day, about 50 mg/day to about 8,000 mg/day, about 50 mg/day to about 2,000 mg/day, about 100 mg/day to about 9,000 mg/day, about 100 mg/day to about 5,000 mg/day, about 100 mg/day to about 4,000 mg/day, and about 100 mg/day to about 2,000 mg/day.

### EXAMPLES

### Example 1—Preparation of Solid Dispersion Formulations with Spray Drying

Spray dried solid dispersion formulations were prepared by solubilizing genistein (8-10 micron agglomerated particles) with polyvinylpyrrolidone in an organic solvent to form a solution. The solutions were stirred until complete dissolution was observed. The solutions were then spray dried, resulting in amorphous solid particles (between about 1-50 microns in size) of genistein dispersed in a polyvinylpyrrolidone matrix. The samples were then further dried in a vacuum oven. The following samples were prepared, with the genistein/polyvinylpyrrolidone being loaded in the solvent at 1.4 wt %:

TABLE 1

| | Genistein | Polyvinylpyrrolidone | |
|---|---|---|---|
| Sample | (% w/w) | (% w/w) | Solvent |
| 1 | 30% | 70% | Methanol |
| 2 | 50% | 50% | Methanol |
| 3 | 60% | 40% | Methanol |
| 4 | 50% | 50% | 2:1 |
| | | | Acetone/Methanol |
| 5 | 40% | 60% | 2:1 |
| | | | Acetone/Methanol |

### Example 2—Preparation of Solid Dispersion Formulations with Hot Melt Extrusion

A hot melt extrusion solid dispersion formulation was prepared by heating a mixture of 35% w/w genistein (8-10 micron agglomerated particles) with 65% w/w polyvinylpyrrolidone at about 200° C. to form a blend of genistein dispersed in melted polyvinylpyrrolidone. The blend was then extruded to form solid pellets of a genistein dispersed in a polyvinylpyrrolidone matrix. The resulting pellets were milled to particle sizes of between about 10 and about 1000 microns.

### Example 3—Comparative PK in Mice

The pharmacokinetics and radioprotective efficacy of the solid dispersion formulations disclosed herein (Sample 1) were compared to the following genistein compositions: Comparative Sample 2—an aqueous nanosuspension of genistein; Comparative Sample 3—a buffered aqueous nanosuspension of genistein; Comparative Sample 4—a lipid nanosuspension of genistein; and Comparative Sample 5—spray dried aqueous nanocrystals of genistein. In contrast to the spray dried solid dispersion, the spray dried aqueous nanocrystals were formed by spray drying an aqueous nanosuspension to generate the spray dried particles. The specific compositions tested were as follows:

TABLE 2

| | | Genistein | | Composition |
|---|---|---|---|---|
| Sample | Description | Concentration | | (% in w/w) |
| Sample 1 | Spray Dried | 0.5 | mg/mg | 50% Polyvinylpyrrolidone |
| | Dispersion | | | |
| Comparative | Aqueous | 325 | mg/mL | 2 mg/mL Polysorbate 80 |
| Sample 2 | Nano- | | | 50 mg/mL Polyvinyl- |
| | suspension | | | pyrrolidone |
| Comparative | Buffered | 250 | mg/mL | 2 mg/mL Polysorbate 80 |
| Sample 3 | Aqueous | | | 50 mg/mL Polyvinyl- |
| | Nano- | | | pyrrolidone |
| | suspension | | | 0.2% Methylparaben |
| | | | | 0.03% Propylparaben |
| | | | | 50 mM Phosphate, pH 7.0 |
| Comparative | Lipid | 220 | mg/mL | 4% Polyvinyl- |
| Sample 4 | Nano- | | | pyrrolidone |
| | suspension | | | 35% Glycerol Trioleate |
| | | | | 35% Corn Oil |
| | | | | 20% Polysorbate 80 |
| | | | | 10% Ethanol |
| Comparative | Spray Dried | 0.49 | mg/mg | 49.7% Polyvinyl- |
| Sample 5 | Nanocrystals | | | pyrrolidone |
| | | | | 0.3% Polysorbate 80 |

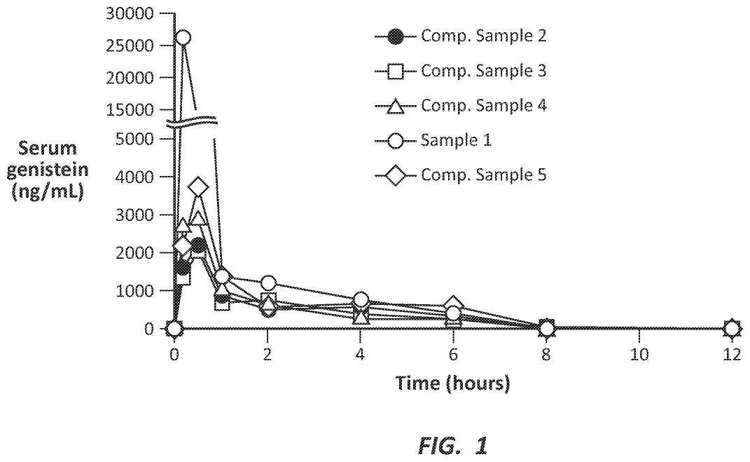

The samples were each orally administered as a single dose (200 mg Genistein/kg) to groups of mice and blood serum concentrations of genistein-aglycone were measured. Drug exposure parameters are shown in Table 3 below as the mean+/−standard deviation. As shown therein, Comparative Samples 2-5 all exhibited similar pharmacokinetic properties. Sample 1, the spray dried solid dispersion exhibited improved bioavailability as evidenced by a 10× increase in Cmax and a 3.5× increase in AUC compared to Comparative Sample 2. The serum genistein measurements of the various samples are also depicted in FIG. 1.

TABLE 3

| | Cmax | Tmax | AUC₀₋₂₄ |
|---|---|---|---|
| Sample | (ng/mL) | (hours) | (ng*hr/mL) |
| Sample 1 | 27467 +/− 5147 | 0.2 +/− 0.14 | 18225 +/− 1970 |
| Comparative | 2547 +/− 588 | 0.4 +/− 0.17 | 5299 +/− 329 |
| Sample 2 | | | |
| Comparative | 2098 +/− 332 | 0.5 +/− 0 | 4813 +/− 366 |
| Sample 3 | | | |
| Comparative | 3302 +/− 761 | 0.4 +/− 0.17 | 5780 +/− 359 |
| Sample 4 | | | |
| Comparative | 3722 +/− 796 | 0.5 +/− 0 | 6629 +/− 486 |
| Sample 5 | | | |

### Example 4—In-vivo Comparison of Prophylactic Efficacy (30-Day)

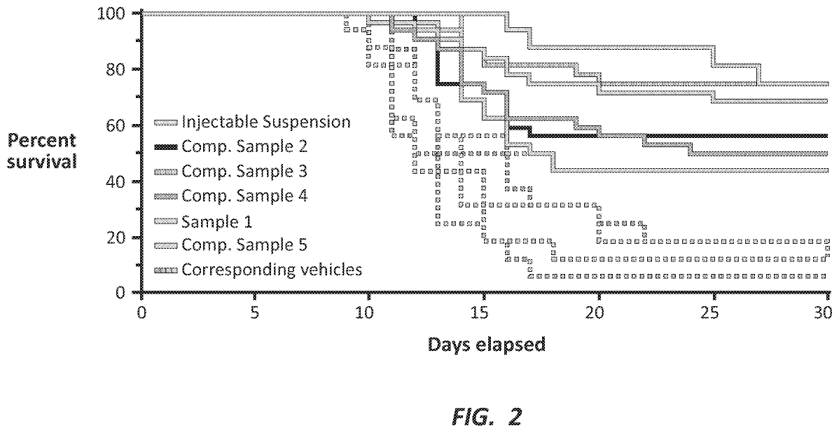

In another study, the samples of Example 3 were each orally administered at 200 mg Genistein/kg twice per day to groups of mice for 6 consecutive days. A separate vehicle control and genistein group was included for each sample formulation. The mice were then exposed to bilateral whole-body irradiation at a dose of 9.2 Gy at 0.6 Gy/min and the thirty-day survival rate was observed. As additional controls, an injectable suspension (200 mg Genistein/kg) and corresponding vehicle were also administered to groups of mice via intramuscular injection 24 hours prior to exposure to bilateral whole-body irradiation at a dose of 9.2 Gy at 0.6 Gy/min. The results of this study are shown in Table 4 as percentage of surviving animals, and the number of surviving animals compared to the total number of animals in the study and depicted in FIG. 2. As shown therein, the survival rate for Sample 1 provided among the highest survival rate of all the samples tested.

TABLE 4

| Sample | 30-Day Survival |
|---|---|
| Sample 1 | 69% (22/32) |
| Comparative Sample 2 | 56% (18/32) |
| Comparative Sample 3 | 75% (24/32) |
| Comparative Sample 4 | 50% (16/32) |
| Comparative Sample 5 | 44% (14/32) |
| Injectable Suspension | 75% (12/16) |

### Example 5—In-Vivo Comparison of Prophylactic Efficacy (180-Day)

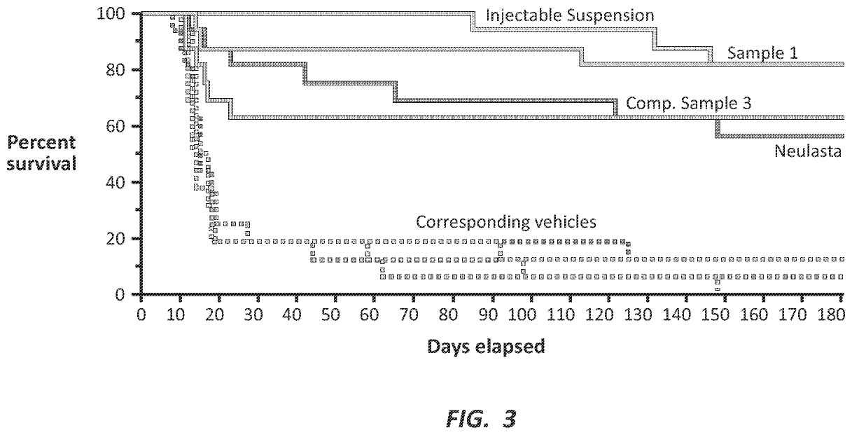

In another study, selected samples of Example 3 were each orally administered at 200 mg Genistein/kg twice per day to groups of mice for 6 consecutive days. A separate vehicle control and genistein group was included for each sample formulation. The mice were then exposed to bilateral whole-body irradiation at a dose of 9.2 Gy at 0.6 Gy/min and the 180-day survival rate was observed. As additional controls, an injectable suspension (200 mg Genistein/kg) and corresponding vehicle were also administered to groups of mice via intramuscular injection 24 hours prior to exposure to bilateral whole-body irradiation at a dose of 9.2 Gy at 0.6 Gy/min. A separate group of mice was also administered a single subcutaneous injection of Neulasta® (0.3 mg/kg), an FDA-approved radiation treatment drug, 24 hours after exposure to bilateral whole-body irradiation at a dose of 9.2 Gy at 0.6 Gy/min. The results of this study are shown in Table 5 as percentage of surviving animals, and the number of surviving animals compared to the total number of animals in the study and depicted in FIG. 3. As shown therein, the survival rate for Sample 1 was surprisingly comparable to the survival rate of the injectable suspension and better than the survival rate of Comparative Sample 3 and Neulasta®.

TABLE 5

| Sample | 180-Day Survival |
|---|---|
| Sample 1 | 81% (13/16) |
| Comparative Sample 3 | 63% (10/16) |
| Injectable Suspension | 81% (13/16) |
| Neulasta ® | 56% (9/16) |

### Example 6—In-Vitro Study of Amorphous Dispersions of Genistein

In another study, the likely impact on bioavailability was studied with the use of different pharmaceutically acceptable excipients. One sample was prepared by spray drying a 30% genistein/70% polyvinylpyrrolidone (w/w) mixture solubilized in methanol to form solid dispersion particles of Sample 6. Another sample was prepared by spray drying a 30% genistein/70% hypromellose acetate succinate (w/w) mixture solubilized in acetone to form solid dispersion particles of Sample 7.

The amorphous solid dispersion particles of Sample 6 and Sample 7 were then each subjected to a gastric-to-intestinal buffer transfer dissolution test. In this test, equivalent amounts of amorphous solid dispersion particles of Sample 6 and Sample 7 were each dissolved in gastric media (0.01 N HCl, pH2) for about 30 minutes. The samples were then transferred to an intestinal buffer (1× phosphate buffered saline (PBS) (67 mM PBS) with 0.5 wt % FaSSIF V1 powder (a fasted state simulated intestinal fluid obtained from biorelevant)) and subjected to an ultracentrifugation assay, where larger-sized particles precipitate with higher speeds. Four general categories of particle size/dynamics were observed: solid aggregates (particles >1 micron), colloids (particles 10-400 nm), micelles (particles 5-50 nm), and unbound drug (particles ˜1 nm). Without being bound by theory, the smaller the particles in the solution, the more available they are for transit across membranes or utilization as a biochemical substrate in vivo. Sample 6 yielded more smaller particles, indicating likely better bioavailability, as shown in the table below.

TABLE 6

| | Micelles Particle | Unbound Drug Particle |
|---|---|---|
| | Distribution | Distribution |
| Sample | (particles 5-50 nm) | (particles ~1 nm) |
| Sample 6 | 171 μg genistein/mL | 57 μg genistein/mL |
| Sample 7 | 50 μg genistein/mL | 17 μg genistein/mL |

### Example 7—Comparative PK in Nonhuman Primates

The pharmacokinetics of an extruded solid dispersion genistein formulation (Sample 8) (similar to the formulation of Example 2) were compared to an aqueous nanosuspension of genistein (Comparative Sample 9). The specific compositions tested were as follows:

TABLE 7

| | | Genistein | | Composition |
|---|---|---|---|---|
| Sample | Description | Concentration | | (% in w/w) |
| Sample 8 | Hot Melt | 354 | mg/g | 65% Polyvinyl- |
| | Extrusion | | | pyrrolidone |
| Comparative | Aqueous | 325 | mg/mL | 2 mg/mL Polysorbate 80 |
| Sample 9 | Nano- | | | 50 mg/mL Polyvinyl- |
| | suspension | | | pyrrolidone |
| | | | | 0.18% Methylparaben |
| | | | | 0.02% Propylparaben |

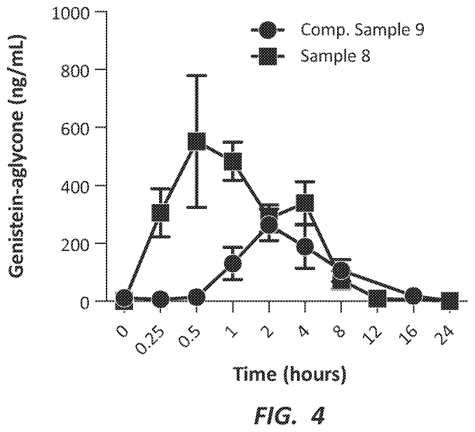

The samples were each orally administered as a single dose (100 mg Genistein/kg) to groups of nonhuman primates and blood serum concentrations of genistein-aglycone were measured. Drug exposure parameters are shown in Table 8 below as the mean+/−standard deviation. As shown therein, Sample 8, the extruded solid dispersion, exhibited improved bioavailability as evidenced by a 2× increase in Cmax and a 1.3× increase in AUC compared to Comparative Sample 9. The serum genistein measurements of the samples are also depicted in FIG. 4.

TABLE 8

| | Cmax | Tmax | AUC₀₋₄₈ |
|---|---|---|---|
| Sample | (ng/mL) | (hours) | (ng*hr/mL) |
| Sample 8 | 663 +/− 165 | 1.00 +/− 0.35 | 2463 +/− 418 |
| Comparative | 311 +/− 117 | 2.50 +/− 1.00 | 1917 +/− 857 |
| Sample 9 | | | |

### Example 8—Comparative PK in Humans

The pharmacokinetics of an extruded solid dispersion genistein formulation containing sucralose (Sample 10) (similar to the formulation of Example 2) were compared to synthetic genistein (BONISTEIN) powder disposed in oral capsules (Comparative Sample 11). The specific compositions tested were as follows:

TABLE 9

| | | Genistein | | Composition |
|---|---|---|---|---|
| Sample | Description | Concentration | | (% in w/w) |
| Sample 10 | Hot Melt | 347 | mg/g | 64.3% Polyvinyl- |
| | Extrusion | | | pyrrolidone |
| | with Sucralose | | | 1% Sucralose |
| Comparative | BONISTEIN in | 500 | mg/capsule | 100% |
| Sample 11 | Oral Capsules | | | BONISTEIN |

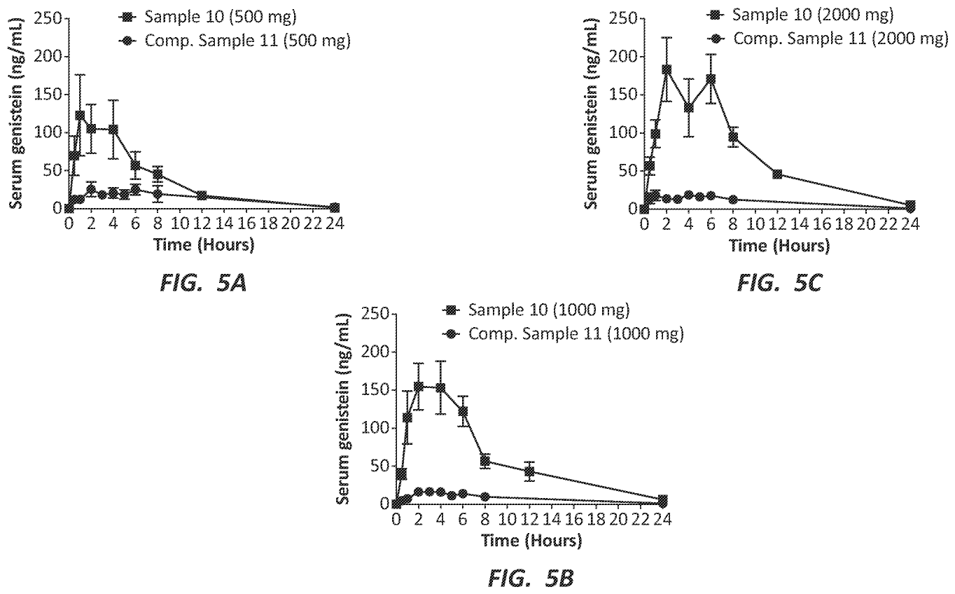

The samples were each orally administered as a single dose (500, 1000 or 2000 mg Genistein) to groups of healthy human volunteers and blood serum concentrations of genistein-aglycone were measured. Drug exposure parameters are shown in Table 10 below as the mean+/−standard deviation. As shown therein, Sample 10, the extruded solid dispersion containing sucralose, exhibited improved bioavailability at doses of 500, 1000 and 2000 mg/genistein as evidenced by a 3.8×, 9.0× and 8.3× increase in Cmax and a 4.5×, 4.1× and 7.8× increase in AUC compared to equivalent doses of Comparative Sample 11, respectively. The serum genistein measurements of the samples are also depicted in FIGS. 5A-5C.

TABLE 10

| | mg | Cmax | Tmax | AUC₀₋₄₈ |
|---|---|---|---|---|
| Sample | Genistein | (ng/mL) | (hours) | (ng*hr/mL) |
| Sample 10 | 500 | 148 +/− 120 | 2.6 +/− 2.1 | 863 +/− 613 |
| Sample 10 | 1000 | 204 +/− 61 | 3.5 +/− 1.8 | 1492 +/− 506 |
| Sample 10 | 2000 | 240 +/− 68 | 3.7 +/− 2.0 | 1669 +/− 367 |
| Comparative | 500 | 38.7 +/− 25.9 | 4.2 +/− 2.4 | 190 +/− 116 |
| Sample 11 | | | | |
| Comparative | 1000 | 22.8 +/− 9.5 | 4.0 +/− 1.7 | 367 +/− 378 |
| Sample 11 | | | | |
| Comparative | 2000 | 28.8 +/− 11.1 | 3.9 +/− 2.9 | 214 +/− 73.6 |
| Sample 11 | | | | |

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A solid dispersion formulation suitable for oral consumption by a subject, the formulation comprising:
genistein; and
one or more pharmaceutically acceptable excipients.

2. The formulation of claim 1, wherein the one or more pharmaceutically acceptable excipients comprises a water soluble polymer.

3. The formulation of claim 2, wherein the water soluble polymer is polyvinylpyrrolidone.

4. The formulation of claim 1, wherein the solid dispersion comprises between about 25% and about 50% (w/w) genistein.

5. The formulation of claim 1, wherein the solid dispersion comprises between about 50% and about 75% (w/w) polyvinylpyrrolidone.

6. The formulation of claim 1, wherein the genistein is dispersed substantially uniformly throughout the solid dispersion.

7. The formulation of claim 1, wherein the solid dispersion comprises particles of between about 1 and about 1000 microns.

8. The formulation of claim 1, wherein the formulation further comprises a non-nutritive sweetener.

9. A method for preparing a solid dispersion formulation comprising genistein, the method comprising:
providing genistein;
providing one or more pharmaceutically acceptable excipients;
solubilizing the genistein and the one or more pharmaceutically acceptable excipients in an organic solvent to form a solution; and
spray drying the solution to form amorphous solid dispersion particles.

10. The method of claim 9, wherein the one or more pharmaceutically acceptable excipients comprises a water soluble polymer.

11. The method of claim 10, wherein the water soluble polymer is polyvinylpyrrolidone.

12. The method of claim 9, wherein the amorphous solid dispersion particles comprise between about 25% and about 50% (w/w) genistein.

13. The method of claim 9, wherein the amorphous solid dispersion particles comprise between about 50% and about 75% (w/w) polyvinylpyrrolidone.

14. The method of claim 9, wherein the genistein is dispersed substantially uniformly throughout the amorphous solid dispersion particles.

15. The method of claim 9, wherein the solid dispersion comprises particles of between about 1 and about 1000 microns.

16. The method of claim 9, wherein the formulation further comprises a non-nutritive sweetener.

17. A method for preparing a solid dispersion formulation comprising genistein, the method comprising:
mixing genistein and one or more pharmaceutically acceptable excipients to form a mixture;
heating the mixture;
extruding the mixture; and
forming an extruded solid dispersion.

18. The method of claim 17, wherein the one or more pharmaceutically acceptable excipients comprises a water soluble polymer.

19. The method of claim 18, wherein the water soluble polymer is polyvinylpyrrolidone.

20. The method of claim 17, wherein the step of heating the mixture comprises heating the mixture to between about 140° C. and about 220° C.
